(19) United States
(12) Patent Application Publication
(10) Pub. No.: US 2024/0325216 A1
(43) Pub. Date: Oct. 3, 2024

(54) NONWOVEN SUBSTRATE AND ABSORBENT ARTICLES COMPRISING THE SAME

(71) Applicant: The Procter & Gamble Company, Cincinnati, OH (US)

(72) Inventors: WEI WU, BEIJING (CN); GUELTEKIN ERDEM, CINCINNATI, OH (US); YIBO FAN, BEIJING (CN)

(21) Appl. No.: 18/617,669

(22) Filed: Mar. 27, 2024

(30) Foreign Application Priority Data
Mar. 30, 2023 (WO) PCT/CN2023/085037

(51) Int. Cl. A61F 13/514; A61F 13/15; A61F 13/511

(52) U.S. Cl. CPC A61F 13/5148; A61F 13/51121; A61F 2013/15292; A61F 2013/15373; A61F 2013/15406; A61F 2013/15447; A61F 2013/15943; A61F 2013/51165; A61F 2013/51452

(57) ABSTRACT

The present disclosure is directed to a nonwoven substrate having a first surface, an opposite second surface, a first layer forming the first surface, and a second layer located below the first layer in the direction of the thickness of the nonwoven substrate. The first layer has a first fiber orientation disorder level, and the second layer has a second fiber orientation disorder level higher than the first fiber orientation disorder level as measured according to Fiber Orientation Disorder Level Test. The present disclosure is also directed to an absorbent article comprising the nonwoven substrate.

### CROSS REFERENCE TO RELATED APPLICATION

This application claims priority to PCT Patent Application Number PCT/CN2023/085037, filed on Mar. 30, 2023, the entire disclosure of which is incorporated herein by reference.

### FIELD OF THE INVENTION

The invention is for a nonwoven substrate and an absorbent article such as a baby diaper, a training pant, a feminine hygiene sanitary napkin and an adult incontinence product which comprises the nonwoven substrate.

### BACKGROUND OF THE INVENTION

Absorbent articles for personal hygiene, such as disposable diapers for infants, training pants for toddlers, adult incontinence undergarments and/or sanitary napkins are designed to absorb and contain body exudates, in particular large quantities of urine, runny bowel movement (BM) and/or menses.

These absorbent articles comprise several layers providing different functions. A liquid permeable topsheet is disposed closest to the wearer's skin and should be capable of quickly absorbing the excreted fluid and providing breathability. A backsheet is disposed on the opposed, garment-facing side of the article. Other components of absorbent articles are well known, and include in particular an absorbent core disposed between the topsheet and the backsheet to absorb and retain the excreted fluids.

A backsheet is a portion touched and observed by the wearer or the caregiver upon use, and thus its properties are most associated with the function and quality of the article. The backsheet typically comprises a liquid impermeable material. Many absorbent articles in the market further comprises a nonwoven outermost layer. A nonwoven outermost layer is generally expected to be soft and contribute cushiony feel to the absorbent article.

Much work has been done to provide loftiness and pleasant tactile sense such as softness and cushiony touch in addition to functional performances such as desirable fluid handling properties, breathability, etc. depending on the desirables of the resulting article. In addition, component layers in an absorbent article are desired to have sufficient mechanical properties such as tensile strength to withstand absorbent article production process, and avoid tearing issues occurred during usage.

Increase of a basis weight of a nonwoven layer can increase mechanical strength of the nonwoven layer as well as provide softness and cushiony feel. However, it brings increase in production cost as well. Carded air-through nonwoven has been known to provide softness with bulkiness in cushioning properties, however, carded air-through method hardly produces low basis weight nonwoven for example, 16 gsm or below since the fiber amount is too less to form a uniform nonwoven web and it results in a low thickness and poor cushioning feel, and decease in material tensile strength especially in a cross machine direction.

Some absorbent articles such as diapers are manufactured to include back cars. For example, some varieties of diapers are manufactured with a pair of oppositely-oriented side back cars attached to an outer cover laterally from each side of a back waist region of the diaper chassis. A back car has a fastener located at or near the outboard edge thereof, and is adapted to attach or adhere to a fastener receiving zone (“landing zone”) disposed on a front waist region of the chassis. While a user applies a diaper on a wearer, the user typically uses a force to pull the back cars to achieve a good sustained fit and minimal leakage with the diaper. If the outer cover does not have a sufficient cross machine direction tensile strength, back cars and/or leg cuffs tend to be torn off from the diaper during user's pulling the back ears and/or leg cuffs, in all probability.

Thus, there is a need for nonwoven with a high loft in a given basis weight.

In addition, there is a need for nonwoven with improved mechanical strength in a given basis weight without compromising sensory feel.

### SUMMARY OF THE INVENTION

The present invention is directed to a nonwoven substrate having a first surface, an opposite second surface, a first layer forming the first surface, and a second layer located below the first layer in the direction of the thickness of the nonwoven substrate, wherein the first layer has a first fiber orientation disorder level, and the second layer has a second fiber orientation disorder level higher than the first fiber orientation disorder level as measured according to Fiber Orientation Disorder Level Test.

The present invention is also directed to an absorbent article comprising the nonwoven substrate disclosed herein.

The article is illustrated in the Figures as a taped diaper. For ease of discussion, the absorbent article and the acquisition-distribution system will be discussed with reference to the numerals referred to in these Figures. The Figures and detailed description should however not be considered limiting the scope of the claims, unless explicitly indicated otherwise. In particular, the invention may also be used in a wide variety of absorbent article forms, such as pant type diapers, which are pre-formed and are worn like an underwear garment, or female protection sanitary pads.

### BRIEF DESCRIPTION OF THE DRAWINGS

FIG. 1 is a schematic view of an exemplary nonwoven disclosed herein.

FIG. 2 is a schematic view of another exemplary nonwoven disclosed herein.

FIGS. 3A-3C are SEM images of a cross-sectional view of an exemplary nonwoven substrate of the present invention.

FIGS. 4A-4C are SEM images of a cross-sectional view of another exemplary nonwoven substrate of the present invention.

FIG. 5 is a schematic plan view of an exemplary absorbent article according to the present invention.

FIG. 6 is a transversal cross-section along 6-6 of the absorbent article of FIG. 5.

FIG. 7 is a perspective view of an exemplary absorbent article according to the present invention.

FIG. 8 is a perspective view of another exemplary absorbent article according to the present invention.

FIG. 9 is a schematic view of specimen preparation for the Cuff Tearing Test disclosed herein.

FIG. 10 is a schematic view of a sample holding fixture according to the Back Ear Testing Test disclosed herein.

FIG. 11A is a fiber density distribution graph in sample nonwoven to determine the main fiber zone according to the Fiber Orientation Disorder Level Test.

FIG. 11B is an illustration to determine a first zone and a second zone according to the Fiber Orientation Disorder Level Test.

FIG. 11C is an illustration to determine fiber orientation disorder level according to the Fiber Orientation Disorder Level Test.

### DETAILED DESCRIPTION OF THE INVENTION

### Definitions of Terms

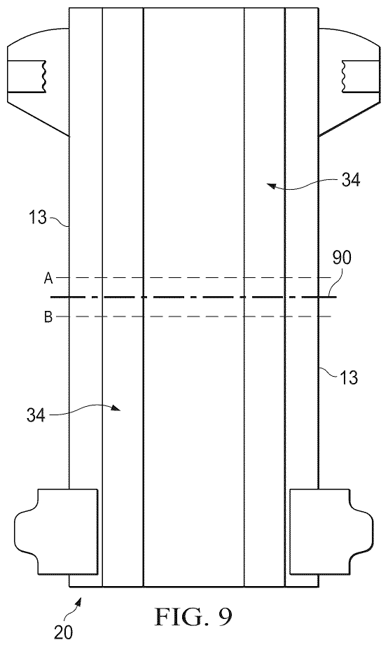

The term “absorbent article” as used herein refers to disposable products such as taped diapers, diapers having a closed waist opening (pants), feminine hygiene sanitary napkins and the like, which are placed against or in proximity to the body of the wearer to absorb and contain bodily exudates such as urine, feces and menses discharged from the body. Typical absorbent articles comprise a topsheet, a backsheet, an absorbent core, an acquisition layer and other components. A liquid permeable topsheet forms at least a portion of the wearer-facing side of the article, and a backsheet forms at least a portion, and typically the whole, of the garment-side of the article. The articles may be provided with fastening elements, such as tapes (taped diapers) or may be provided already pre-formed with a waist opening and a pair of leg openings as in an underwear (pant diapers). The absorbent articles may be for use with babies, infants, women or incontinent adults. Typical features of absorbent articles are further discussed further below, and in relation with the illustrated taped diaper in FIGS. 8 and 9, which is of course for illustration purpose only and not limiting the scope of the inventions, unless specifically indicated otherwise.

The term “nonwoven” as used herein refers to a manufactured material, web, sheet or batt of directionally or randomly oriented fibers, bonded by calendar bonding, and/or through-air bonding, and/or friction, and/or cohesion and/or adhesion, excluding paper and products which are woven, knitted, tufted, stitch-bonded, incorporating binding yarns or filaments, or felted by wet milling, whether or not additionally needled. The fibers may be of natural or man-made origin. The fibers may be staple or continuous filaments or be formed in situ. The porous, fibrous structure of a nonwoven may be configured to be liquid permeable or impermeable, as desired.

“Comprise,” “comprising,” and “comprises” are open ended terms, each specifies the presence of the feature that follows, e.g. a component, but does not preclude the presence of other features, e.g. elements, steps, components known in the art or disclosed herein. These terms based on the verb “comprise” should be read as encompassing the narrower terms “consisting essential of” which excludes any element, step or ingredient not mentioned which materially affect the way the feature performs its function, and the term “consisting of” which excludes any element, step, or ingredient not specified. Any preferred or exemplary embodiments described below are not limiting the scope of the claims, unless specifically indicated to do so. The words “typically”, “normally”, “advantageously” and the likes also qualify features which are not intended to limit the scope of the claims unless specifically indicated to do so.

“Machine direction” or “MD” is the direction parallel to the direction of travel of the web in a manufacturing process. The machine direction is typically the longitudinal direction of a component, such as an ear of an absorbent article. The “cross machine direction” or “CD” is the direction substantially perpendicular to the MD and in the plane generally defined by the web.

### Nonwoven Substrate

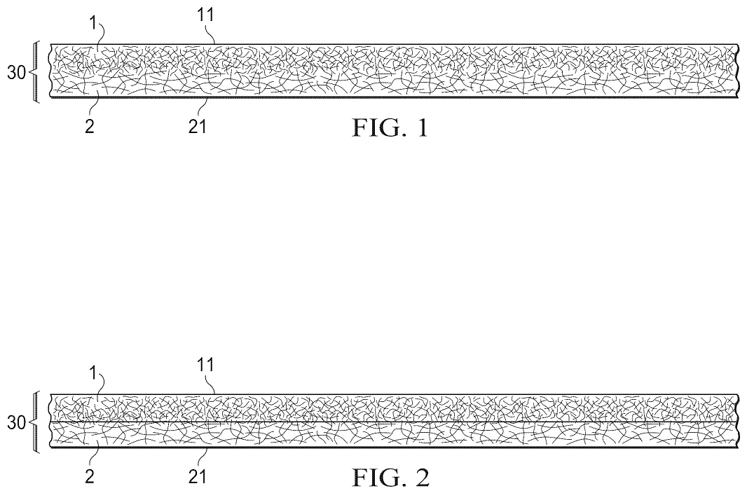

Referring to FIGS. 1 and 2, the nonwoven substrate (herein also more simply referred to as “the nonwoven”) according to the present invention herein has a first surface 11, an opposite second surface 21 and comprises a first layer 1 forming the first surface 11, and a second layer 2 located below the first layer 1 in a z-direction (direction of the thickness of the nonwoven substrate 30). The first layer 1 comprises first fibers and has a first fiber orientation disorder level. The second layer 1 comprises second fibers, and has a second fiber orientation disorder level, the second fiber orientation disorder level being higher than the first fiber orientation disorder level.

Fiber orientation disorder level (“FODL”) is measured according to the Fiber Orientation Disorder Level Test disclosed herein. The difference between the first and second fiber orientation disorder levels may be no less than about 0.05, or no less than about 0.06, or no less than 0.065, or no less than 0.07. The second FODL may be no less than about 0.27, or no less than about 0.28, or no less than about 0.29. Without being bound by theory, with the configuration of a nonwoven substrate having a second FODL is higher than a first FODL at least about 0.05, and a second FODL is no less than about 0.27, the nonwoven substrate can provide balanced softness such as smoothness, cushiony feel and increase thickness, and increased tensile strength.

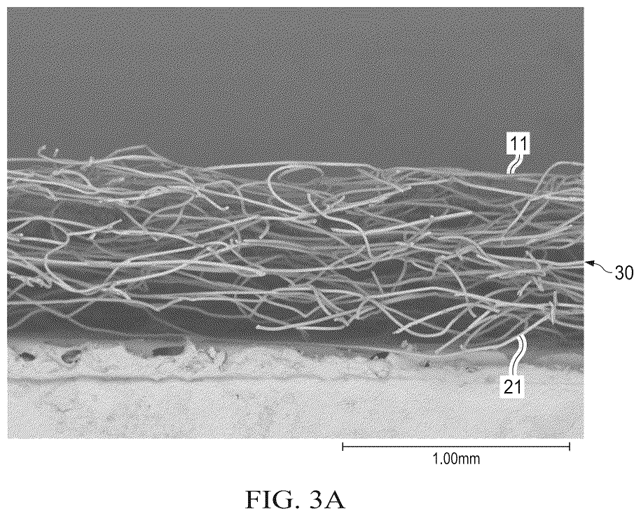

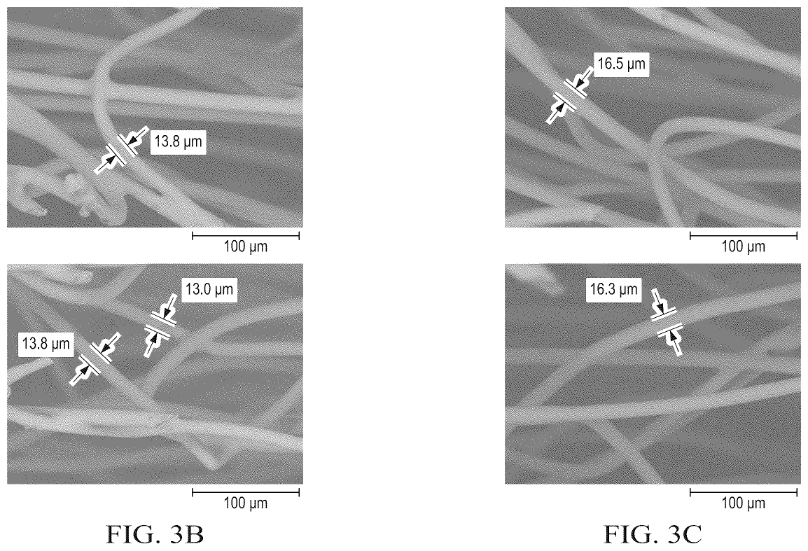

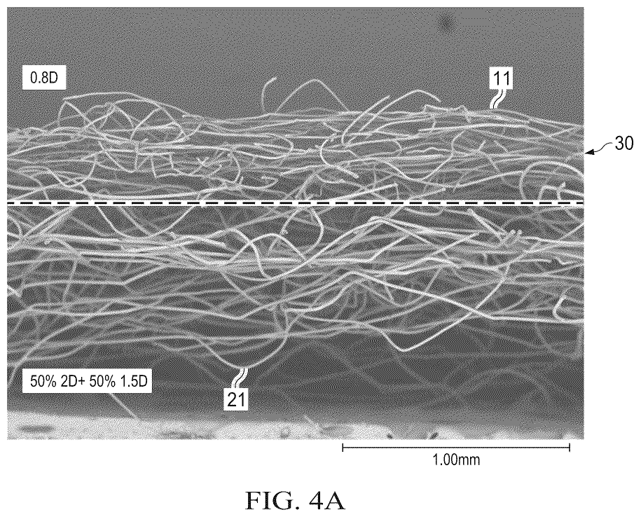

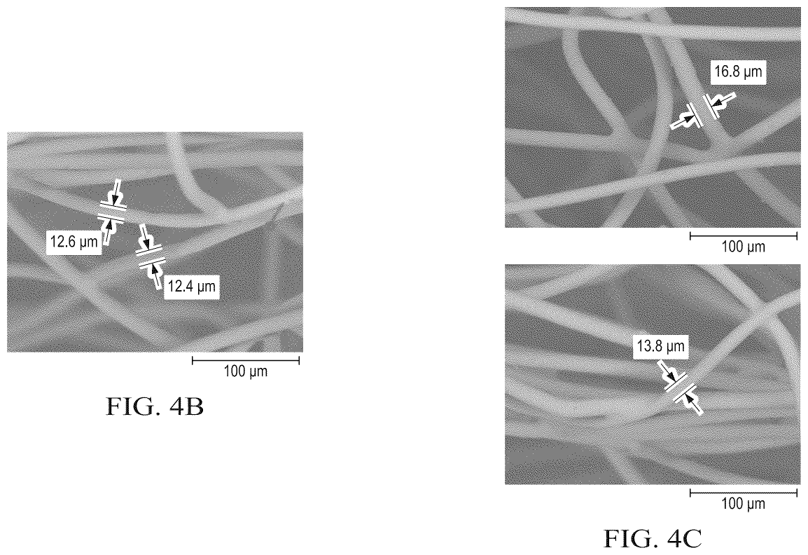

Referring to FIG. 1, FIG. 3A and FIG. 4B, when the nonwoven substrate of the present invention comprises two or more layers, the two or more layers may form a unitary structure or may remain as discrete layers. A unitary structure herein intends to mean that although it may be formed by several sub-layers or stratums that have distinct properties and/or compositions from one another, they are somehow intermixed at the boundary region so that, instead of a definite boundary between sub-layers, it would be possible to identify a region where the different sub-layers transition one into the other. Such a unitary structure is typically built by forming the various sub-layers one on top of the other in a continuous manner, for example using carded, or air laid or wet laid deposition. Or, each of sub-layer is produced in a separate step and the sub-layers are combined together in a face to face relationship. The sub-layers can be integrated via a known integration or bonding process such as spunlacing processes, hydroentangling, calendar bonding, through-air bonding and resin bonding.

Typically, there is no adhesive used between the sub-layers of the unitary material. However, in some cases, adhesives and/or binders can be present although typically in a lower amount.

When the nonwoven substrate of the present invention is described herein, terms of layer(s), sub-layer(s), and stratum (strata) are interchangeable. Relating to descriptions of a nonwoven substrate with unitary structure, the terms of layer(s) and stratum(s) are interchangeable.

Referring to FIG. 2, when the nonwoven substrate of the present invention comprises two or more layers which remain as discrete layers, the two or more layers may be attached at least partially to each other by, for example, thermal bonding, adhesive bonding, ultrasonic bonding, or any combination thereof.

In some embodiments, referring to FIGS. 3A and 4A, the nonwoven substrate of the present invention has a unitary structure. FIG. 3A is a cross section image of an exemplary nonwoven substrate of the present invention having a first layer and a second layer both of which are made by 60% 2d PE/PET sheath/core bicomponent fibers and 40% 1.5d PE/PET sheath/core bicomponent fibers. FIG. 4A is a cross section image of an exemplary nonwoven substrate of the present invention having a first stratum made by 0.8 denier PE/PET sheath/core bicomponent fibers and a second stratum made by 50% 2.0 denier PE/PET fibers PE/PET sheath/core bicomponent fibers and 50% 1.5 denier PE/PET fibers PE/PET sheath-core bicomponent fibers. The nonwoven 30 has unitary structure, and fibers from the first stratum and fibers from the second stratum are intermixed in a boundary region instead of having a definite boundary between the two stratums.

Referring to FIG. 2, nonwoven 30 of the present invention may be a laminate of at least two layers, a first layer 1 and a second layer 2 in this case, which are joined to each other in a face to face relationship.

The first fibers and the second fibers may be staple heat-fusible fibers.

The first fibers and the second fibers may be the same, substantially the same, or different in size, shape, composition, denier, fiber diameter, fiber length, and/or weight. In one embodiment, the first fibers and the second fibers are identical in shape, composition, denier, fiber diameter and fiber length. In another embodiment, the first fibers and the second fibers are different in at least one of shape, composition, denier, fiber diameter and fiber length. For example, the first fibers have a diameter smaller than the diameter of the second fibers. With such an example, when the nonwoven substrate is used as a component layer in an absorbent article in such a way the first layer of the nonwoven substrate forms at least part of the garment-facing surface or the wearer-facing surface of the absorbent article, the absorbent article having overall improved softness connoting high quality and still having a desirable thickness and soft cushiony feel may be obtained.

When a nonwoven substrate is used as an outer cover of an absorbent article, a tensile strength, especially the CD tensile of the outer cover is important in order to avoid a cuff tearing problem and/or a back car tearing problem. The nonwoven substrate of the present invention may have a CD tensile strength no less than about 5.5 N/5 cm, or no less than about 5.8 N/5 cm, or no less than about 6.0 N/5 cm as measured by the Tensile Strength Test disclosed herein. The nonwoven substrate of the present invention may have a CD tensile factor (CD tensile strength per 10 gsm of basis weight) no less than 0.35, or no less than 0.36, or no less than 0.37. Without being bound by theory, with a second layer comprising more disordered fibers, the nonwoven substrate of the present invention can achieve an increased CD tensile strength, and a high CD tensile strength factor without basis weight increase.

In one embodiment, when users apply absorbent articles having cuffs such as leg cuffs, users tend to pull the cuffs to make the cuff standing up. If an outer cover in a diaper does not have sufficient tensile strength especially CD tensile strength, pulling cuffs during application of the diaper causes cuff tearing from the diaper. In another embodiment when users apply a taped diaper having back cars attached to a chassis to a wearer, users use a force to pull the back cars having tape tabs to attach or adhere the tape tabs to a landing zone disposed on a front waist region of the chassis to achieve a good sustained fit and minimal leakage with the diaper. If an outer cover does not have a sufficient CD tensile strength, pulling the back cars tends to cause a back car to be torn from the chassis, the main body of the diaper. An outer cover comprising the nonwoven substrate of the present invention has an increased CD tensile strength even in a low basis weight, and can prevent such tearing problems.

The basis weight of the nonwoven substrate may be no greater than about 45 gsm, or in the range of about 13 gsm to about 45 gsm, about 15 gsm to about 43 gsm, or about 16 gsm to about 40 gsm. As described previously, the nonwoven substrate of the present invention exhibits a high CD tensile strength factor, it can provide a CD tensile strength no less than 5.5 N/5 cm, as measured by Tensile Strength Test even in a quite low basis weight such as about 16 gsm.

The nonwoven substrate of the present invention may have a thickness no less than about 250 μm, or no less than 300 μm, or no less than 350 μm or above as measured by Thickness Test disclosed herein.

A nonwoven substrate when used as a component of an absorbent articles, the thickness of the nonwoven substrate is important to deliver a soft cushiony feel. The nonwoven substrate of the present invention can exhibit a thickness factor (thickness per 10 gsm of basis weight) no less than about 0.25, or no less than about 0.26, or no less than about 0.27, so that the nonwoven can have a high thickness without basis weight increase. Without being bound by theory, with a second layer comprising increased numbers of disordered fibers, the nonwoven substrate of the present invention can achieve an increased thickness, and a high thickness factor.

In some instances, the first layer and the second layer have the same basis weight.

In other instances, it may be desirable the second layer has a higher basis weight than the first layer. For instance, the second layer's basis weight may be at least about 1.1 to about 4 times, at least about 2 to about 3.5 times, about 2 to about 5 times, or about 3 times to about 4 times greater than the first layer's basis weight. By providing the second layer with a higher basis weight than the first layer, it can achieve better material cushiony feel and higher CD tensile since the second layer comprising more disordered fibers may enable the nonwoven substrate the nonwoven substrate of the present invention to exhibit a high CD tensile strength factor and a high thickness factor. As noted previously, a second layer comprising more disordered fibers may enable the nonwoven substrate the nonwoven substrate of the present invention to exhibit a high CD tensile strength factor and a high thickness factor.

The basis weight of the nonwoven substrate, and the stratums may be designed for specific product requirements.

The nonwoven substrate of the present invention may comprise a third layer between the first layer and the second layer, or below the second layer in the thickness direction of the nonwoven substrate. The first layer and the second layer are described in further detail below.

### First Layer

The first layer comprises first fibers, and has a first FODL as measured according to the Fiber Orientation Disorder Level Test. The first layer may have a first FODL no greater than about 0.35. With a first layer with a low FODL, the nonwoven substrate of the present invention may exhibit better surface smoothness and result in better slipperiness.

The first layer may comprise third fibers.

The first fibers may comprise bicomponent fibers such as a sheath/core type and a side-by-side type. The sheath may comprise polyolefin such as polyethylene and the core may comprise polyethylene terephthalate (PET). The sheath and the core may also comprise any other suitable materials known to those of skill in the art. The sheath and the core may each comprise about 50% of the fibers by weight of the fibers, although other variations (e.g., sheath 60%, core 40%; sheath 30%, core 70% etc.) are also within the scope of the present disclosure. The first fibers or other fibers that make up the first layer may have a denier in the range of about 0.5 to about 4, about 0.8 to about 4, about 1.0 to about 4, about 1.5 to about 4, about 1.5 to about 3, about 1.5 to about 2.5, or about 2, specifically including all 0.1 denier increments within the specified ranges and all ranges formed therein or thereby. Denier is defined as the mass in grams per 9000 meters of a fiber length. The denier of the first fibers may be in the range of about 0.5 denier to about 4 denier, or about 0.6 denier to about 4 denier, or about 0.6 denier to about 2 denier.

The first fibers or other fibers that make up the first layer may have a diameter in the range of about 7 μm to about 24 μm, about 9 μm to about 22 μm, about 11 μm to 20 μm, specifically including all 0.1 denier increments within the specified ranges and all ranges formed therein or thereby.

The optional third fibers making up the first layer may be bicomponent fibers, or any other suitable types of fibers, such as polypropylene fibers, other polyolefins, other polyesters besides PET such as polylactic acid, thermoplastic starch-containing sustainable resins, other sustainable resins, bio-PE, bio-PP, and Bio-PET, viscose fibers, rayon fibers, or other suitable nonwoven fibers, for example. These fibers may have any suitable deniers or denier ranges and/or fiber lengths or fiber length ranges.

The basis weight of the first layer may be in the range of about 6 gsm to about 23 gsm, about 8 gsm to about 20 gsm, or about 10 gsm to about 20 gsm.

The first layer may have a thickness no less than about 0.1 μm, no less than about 0.2 μm or 0.4 μm as measured by Thickness Test disclosed herein.

### Second Layer

The second layer comprises second fibers, and has a second FODL. The second FODL may be no less than about 0.27, or no less than about 0.28, or no less than about 0.29.

As noted previously, a second layer comprising fibers more disordered than fibers making up the first layer may enable the nonwoven substrate of the present invention to exhibit a high CD tensile strength factor and a high thickness factor.

The second fibers or other fibers that make up the second layer may have a denier in the range of about 0.5 to about 4, about 0.8 to about 4, about 1.0 to about 4, about 1.5 to about 4, about 1.5 to about 3, about 1.5 to about 2.5, or about 2, specifically including all 0.1 denier increments within the specified ranges and all ranges formed therein or thereby. The denier of the second fibers may be in the range of about 1 denier to about 4 denier, or about 1.2 denier to about 3.5 denier, or about 1.5 denier to about 3 denier. The second fibers or other fibers that make up the second layer may have a diameter in the range of about 11 μm to about 24 μm, about 12 μm to about 22 μm, about 14 μm to 20 μm, as measured by Fiber Diameter Test disclosed herein, specifically including all 0.1 denier increments within the specified ranges and all ranges formed therein or thereby.

The basis weight of the second layer may be in the range of about 6 gsm to about 30 gsm, about 10 gsm to about 25 gsm, or about 12 gsm to about 22 gsm.

The second layer may comprise fourth fibers. The fourth fibers may have a linear density and/or a fiber diameter smaller than those of the second fibers. By blending fourth fibers having a smaller linear density or a smaller fiber diameter, the second layer can have increased number of fibers without increasing basis weight, so that the second layer can have increased number of heat-fused points, which results better cushiony feel and higher material tensile especially for CD tensile.

The second fibers may comprise bicomponent fibers such as a sheath/core type and a side-by-side type. The sheath may comprise polyolefin such as polyethylene and the core may comprise polyethylene terephthalate (PET). The sheath and the core may also comprise any other suitable materials known to those of skill in the art. The sheath and the core may each comprise about 50% of the fibers by weight of the fibers, although other variations (e.g., sheath 60%, core 40%; sheath 30%, core 70% etc.) are also within the scope of the present disclosure. The second fibers or other fibers that make up the second layer may have a denier in the range of about 1 to about 6, about 1.5 to about 4, about 1.0 to about 4, about 1.5 to about 3, about 1.5 to about 2.5, specifically including all 0.1 denier increments within the specified ranges and all ranges formed therein or thereby.

When the second layer comprises optional fourth fiber, in certain instances, the denier of the fourth staple heat-fusible fibers may be lower than the denier of the second fibers. By providing the fourth fibers with a denier lower than a denier of the second fibers of the second layer, the second layer can have increased number of fibers without increase in a basis weight, so that the second layer has increased number of heat-fused points which may result better cushiony fell and higher material tensile especially for CD tensile.

The optional fourth fibers in the second layer may be bicomponent fibers, or any other suitable types of fibers, such as polypropylene fibers, other polyolefins, other polyesters besides PET such as polylactic acid, thermoplastic starch-containing sustainable resins, other sustainable resins, bio-PE, bio-PP, and Bio-PET, viscose fibers, rayon fibers, or other suitable nonwoven fibers, for example. These fibers may have any suitable deniers or denier ranges and/or fiber lengths or fiber length ranges.

### Process for Nonwoven Production

The nonwoven substrate of the present invention can be produced by processes well known in the nonwoven industry. For example, the nonwoven substrate can by produced by carding process and subsequent heat-bonding process.

Carding is a mechanical process whereby clumps of staple fibers are separated into individual fibers and simultaneously made into a coherent web. Carding is typically carried out on a machine that utilizes opposed moving beds or surfaces of fine, angled, closely spaced teeth or wires or their equivalent to pull and tease the clumps apart. The teeth of the two opposing surfaces typically are inclined in opposite directions and move at different speeds relative to each other. which made fibers orientation disorder level as designed. A nonwoven web may be formed in a subsequent laydown step.

Heat-bonding of fibers can be conducted using a known process in the nonwoven industry such as air-through bonding. Air-through bonding means a process of bonding fibers by forcing air through the nonwoven web, wherein the air is sufficiently hot to melt (or at least partly melt, or melt to a state where the fiber surface becomes sufficiently tacky) the polymer of a fiber or, if the fibers are multicomponent fibers, wherein the air is sufficiently hot to melt (or at least partly melt, or melt to a state where the fiber surface becomes sufficiently tacky) one of the polymers of which the fibers of the nonwoven web are made. The melting and re-solidification of the polymer provide the bonding between different fibers.

### Absorbent Article

An absorbent article of the present invention comprises a liquid pervious topsheet, a liquid impervious backsheet, an absorbent core disposed between the topsheet and the backsheet, and a nonwoven substrate disclosed herein.

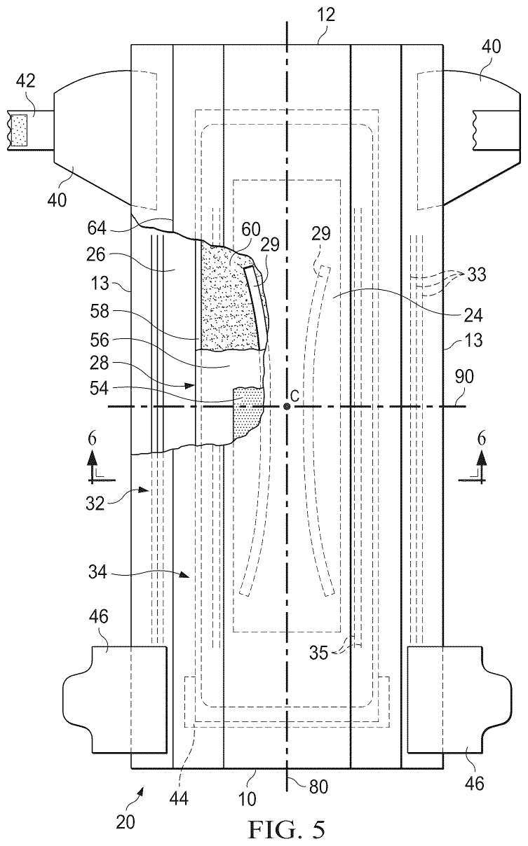

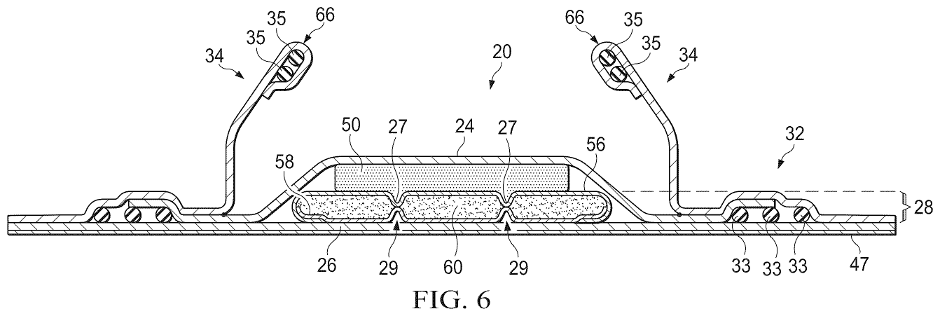

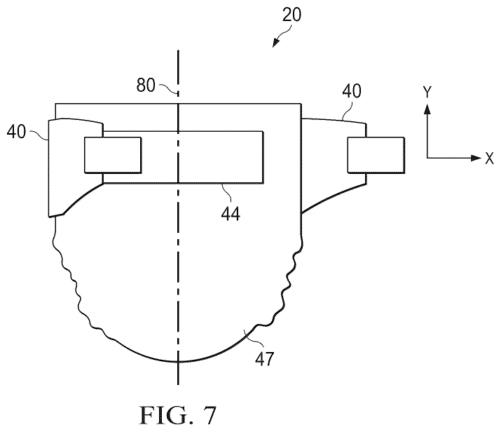

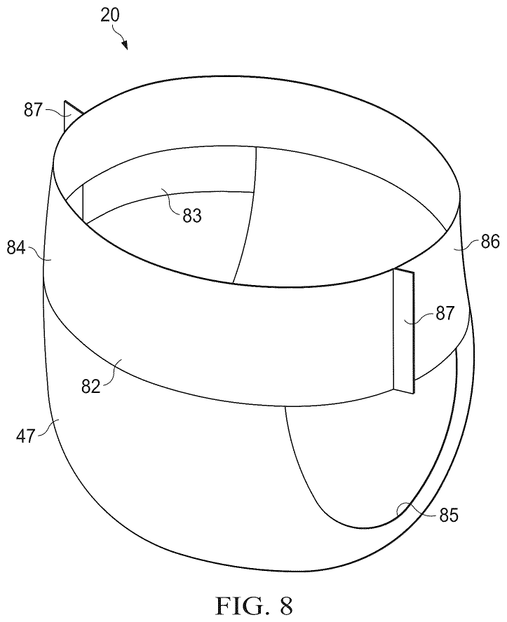

Absorbent articles will now be generally discussed and further illustrated in the form of a taped type baby diaper 20 as exemplarily represented in FIGS. 5-7. FIG. 5 is a plan view of the exemplary diaper 20, a taped type diaper, in a flattened-out configuration with the taped ends opened and the wearer-facing side turned up. FIG. 6 is a transversal cross-section along 6-6 of the absorbent article of FIG. 5. FIG. 6 is a schematic perspective view of a taped type diaper. As said, this diaper 20 is shown for illustration purpose only as the structure of the present invention may be comprised in a wide variety of diapers or other absorbent articles, such as pants.

Referring to FIGS. 5-7, an absorbent article typically has a front edge 10, a back edge 12 and the longitudinally-extending side edges 13. The front edge 10 forms the edge of the front waist and the back edge 12 of the back waist, which together when worn by the wearer form the opening for the waist of the wearer. The side edges 13 can each form one of the leg openings. The absorbent article 20 notionally comprises a longitudinally centerline 80 dividing the article in a left side and a right side, and a perpendicular transversal centerline 90 disposed at half the length of the article as measured on the longitudinal centerline 80, with both centerlines crossing at the center point C.

Other layers of the absorbent article are better illustrated in FIG. 6, which shows in cross-section in addition to the liquid permeable topsheet 24 and the backsheet 26, an absorbent core 28 between the topsheet 24 and the backsheet 26.

An optional acquisition and distribution system (“ADS”) 50 is represented in FIG. 6 together with other typical diaper components. The ADS may comprise one layer or more than one layer. Typical ADS may not comprise SAP as this may slow the acquisition and distribution of the fluid, but an additional layer may also comprise SAP if some fluid retention properties are wished.

The absorbent article 20 may comprise a pair of partially upstanding barrier leg cuffs 34 having clastic elements 35 and elasticized gasketing cuffs 32 having elastic elements 33 substantially planar with the chassis. Both types of cuffs are typically joined to the chassis of the absorbent article typically via bonding to the topsheet and/or backsheet.

FIG. 8 is a perspective view of another type of diaper, a pant type diaper. Referring to FIG. 8, diaper 20 has a wearer-facing surface and a garment-facing surface, and comprises a front elastic belt region 84, a back elastic belt region 86, a crotch region 85, and a pair of side seams 87 which join the front elastic belt region 84 and the back elastic belt region 86 to form a waist opening and a pair of leg openings. Each of the front and back elastic belt region comprises a laminate comprising an inner belt sheet 83, an outer belt sheet 82, and a plurality of elastic members running in the transverse direction. The outer belt sheet 82 can comprise the nonwoven substrate of the present invention in such a way that the first layer of the nonwoven substrate forms the garment-facing surface of the absorbent article.

### Topsheet

A suitable topsheet may be manufactured from a wide range of materials, such as porous foams, reticulated foams, apertured plastic films, woven materials, woven or nonwoven materials of natural fibers (e.g., wood or cotton fibers), synthetic fibers or synthetic filaments (e.g., polyester or polypropylene or bicomponent PE/PP fibers or mixtures thereof), or combinations thereof.

When the topsheet 24 comprises the nonwoven substrate disclosed herein, the first layer of the nonwoven substrate is oriented outwardly relative to the absorbent article 20, so that the first layer can be felt by the caretaker or a user feeling the wearer-facing side of the article 20.

### Absorbent Core

As used herein, the term “absorbent core” refers to a component used or intended to be used in an absorbent article and which comprises an absorbent material and optionally a core wrap. As used herein, the term “absorbent core” does not include the topsheet, the backsheet and any acquisition-distribution layer or multilayer system, which is not integral part of the absorbent core. The absorbent core is typically the component of an absorbent article that has the most absorbent capacity of all the components of the absorbent article. The terms “absorbent core” and “core” are herein used interchangeably.

Referring to FIGS. 5 and 6, the absorbent core 28 can absorb and contain body exudates received by the absorbent article. The absorbent core 28 may comprises an absorbent material 60, which may be cellulose fibers, a blend of superabsorbent polymers and cellulose fibers, pure superabsorbent polymers, and/or a high internal phase emulsion foam, and a core wrap enclosing the absorbent material. The absorbent core 28 may comprise absorbent material free channels 29, through which the top side 56 of the core wrap may be bonded to the bottom side 58 of the core wrap. Channels may be useful for allowing the absorbent core 28 to bend upon swelling with fluids, such that the absorbent article conforms to the wearer's body after swelling and prevent sagging of the article. The core wrap bonds 27 may at least persist as the absorbent core 28 swells upon liquid absorption and creates three-dimensional channels at the wearer-facing surface of the article. Of course, this is entirely optional, the absorbent core may also not have bonded channels, or even unbonded channels. The absorbent material defines an absorbent material area, which may be rectangular as show in in FIG. 6, but it is also common to have a shaped area which is tapered in the area around the transversal centerline 90.

The absorbent material comprises a liquid-absorbent material commonly used in disposable absorbent articles such as comminuted wood pulp, which is generally referred to as airfelt or fluff. Examples of other suitable liquid-absorbent materials include creped cellulose wadding; melt blown polymers, including co-form; chemically stiffened, modified or cross-linked cellulosic fibers; tissue, including tissue wraps and tissue laminates, absorbent foams, absorbent sponges, superabsorbent polymers (herein abbreviated as “SAP”), absorbent gelling materials, or any other known absorbent material or combinations of materials.

The absorbent material in the absorbent core can be any type. It can be an airfelt core comprising wood cellulose fibers such as pulp fibers mixed with SAP, or an airfelt-free core free from such cellulose fibers. Airfelt cores typically comprises from 40% to 80% of SAP. For absorbent cores comprising a relatively high proportion of SAP at least partially enclosed within the core wrap, the SAP content may represent in particular at least 80%, 85%, 90%, 95% and up to 100%, of superabsorbent polymer by weight of the absorbent material. The absorbent material may in particular comprise no or only small amount of cellulose fibers, such as less than 20%, in particular less than 10%, 5% or even 0% of cellulose fibers by weight of the absorbent material. The absorbent core may comprise an absorbent material comprising at least 80%, at least 90%, at least 95%, or at least 99% by weight of the absorbent core. The term “superabsorbent polymer” refers herein to absorbent material, which may be cross-linked polymer, and that can typically absorb at least 10 times their weight of an aqueous 0.9% saline solution as measured using the Centrifuge Retention Capacity (CRC) test (EDANA method WSP 241.2-05E). The SAP may in particular have a CRC value of more than 20 g/g, or more than 24 g/g, or of from 20 to 50 g/g, or from 20 to 40 g/g, or from 24 to 30 g/g. The SAP may be typically in particulate forms (superabsorbent polymer particles), but it is not excluded that other forms of SAP may be used such as a superabsorbent polymer foam for example.

The absorbent core 28 may comprise an absorbent layer having superabsorbent polymers disposed between first and second layers of nonwoven material immobilized by a fibrous layer of thermoplastic adhesive material (not shown). The first and second layers of nonwoven materials may be relatively low basis weight nonwoven fibrous webs including synthetic fibers, such as mono-constituent fibers of PE, PET and PP, multiconstituent fibers such as side by side, core/sheath or island in the sea type fibers. Such synthetic fibers may be formed via a spunbonding process or a meltblowing process. Such an embodiment is exemplarily shown in FIG. 6. Absorbent layers may be disposed in plurality in the absorbent core 28.

Alternatively, the absorbent core 28 may comprise a high loft material encompassing superabsorbent polymers. The term “high loft” refers to low density bulky fabrics, as compared to flat, paper-like fabrics. High loft webs are characterized by a relatively high porosity. This means that there is a relatively high amount of void space in which superabsorbent polymer particles can be distributed. The high loft material (without the superabsorbent particles) of the invention may have a density at a pressure of 4.14 kPa (0.6 psi) below 0.20 g/cm³, in particular ranging from 0.05 g/cm³ to 0.15 g/cm³. The high loft layer (without the superabsorbent particles) may have a density at a pressure of 2.07 kPa (0.3 psi) below 0.20 g/cm³, in particular ranging from 0.02 g/cm³ to 0.15 g/cm³. The high loft layer (without the superabsorbent particles) may have a density at a pressure of 0.83 kPa (0.12 psi) below 0.15 g/cm³, in particular ranging from 0.01 g/cm³ to 0.15 g/cm³, and a basis weight of from 15 to 500 gsm, preferably 30˜200 gsm, such as those described in US 2021/0361497 A1. The absorbent core 28 comprising high loft material encompassing superabsorbent polymers may also contain channels.

### Backsheet

An absorbent article 20 according to the present invention comprises a liquid impervious backsheet 26. The backsheet may be designed to prevent the exudates absorbed by and contained within the absorbent article 20 from soiling articles that may contact the absorbent article, such as bed sheets and undergarments. The backsheet may be substantially water-impermeable. Suitable backsheet materials may include breathable materials that permit vapors to escape from the absorbent article while still preventing exudates from passing through the backsheet. The backsheet may comprise a liquid impermeable film. The backsheet may comprise a wetness indicator.

### Outer Cover

Referring to FIGS. 6-8, an absorbent article 20 according to the present invention may further comprise an outer cover 47 forming at least part of a garment-facing surface of the absorbent article. When the outer cover 47 comprises a nonwoven substrate, a backsheet 26 and the outer cover 47 may be disposed in a face to face relationship in such a way that the outer cover 47 is towards the garment-facing side of the article, and the backsheet 26 is towards an absorbent core 28 of the article 20.

Exemplary nonwoven substrate suitable for the outer cover 47 includes: air-through carded nonwoven having a thickness of at least about 400 μm, or at least about 450 μm, or at least about 500 μm. Such material may provide a soft lofty feeling to the garment-facing side. Suitable for the outer cover 47 nonwoven of the present invention are air-through carded nonwoven material made of bicomponent fibers.

One non-limiting material for the outer cover 47 is the nonwoven substrate of the present invention. When the nonwoven substrate of the present invention is utilized as the outer cover 47, it may be used in such a way the first layer of the nonwoven substrate forms at least part of the garment-facing surface of the absorbent article, so that the first layer can be felt by the caretaker or a user feeling overall improved softness connoting high quality and still having a desirable soft cushiony feel.

Exemplary nonwoven substrates of the present invention for the outer cover comprises a first layer comprising first fibers of from about 0.6 to about 1 denier, and the second layer comprising second fibers of from about 2 to about 4 denier, and third fibers of lower denier than the second fibers. The second layer has a FODL no less than about 0.27.

### Fastening System

The absorbent article 20 may include a fastening system. The fastening system may be used to provide lateral tensions about the circumference of the absorbent article to hold the absorbent article on the wearer as is typical for taped diapers. This fastening system may not be necessary for training pant articles since the waist region of these articles is already bonded. The fastening system may comprise a fastener such as tape tabs 42 such as adhesive tape tabs or tape tabs comprising hook elements, cooperating with a landing zone 44 (e.g. a nonwoven web providing loops in a hook and loop fastening system).

### Front and Back Ears

The absorbent article 20 may comprise front cars 46 and back ears 40. The cars may be an integral part of the chassis, such as formed from the topsheet 24, backsheet 26 and/or outer cover 47 as side panels. Alternatively, as represented on FIG. 1, the ears may be separate elements attached by gluing, heat embossing, and/or pressure bonding. The back cars 40 may be stretchable to facilitate the attachment of the tabs 42 to the landing zone 44 and maintain the taped diapers in place around the wearer's waist. The back ears 40 may also be elastic or extensible to provide a more comfortable and contouring fit by initially conformably fitting the absorbent article to the wearer and sustaining this fit throughout the time of wear well past when absorbent article has been loaded with fluids or other bodily exudates since the elasticized cars allow the sides of the absorbent article to expand and contract.

### Elastic Waist Feature

The absorbent article 20 may also comprise at least one elastic waist feature (not represented) that helps to provide improved fit and containment. The elastic waist feature is generally intended to elastically expand and contract to dynamically fit the wearer's waist. The elastic waist feature may extend at least longitudinally outwardly from at least one waist edge of the absorbent core 28 and generally forms at least a portion of the end edge of the absorbent article 20. Disposable diapers may be constructed so as to have two elastic waist features, one positioned in the front waist region and one positioned in the rear waist region.

The absorbent article may also comprise other typical components, which are not represented, such as transverse barrier cuff(s), a lotion application, etc.

Components of the disposable absorbent article described in this specification can at least partially be comprised of bio-sourced content as described in US 2007/0219521A1 Hird et al published on Sep. 20, 2007, US 2011/0139658A1 Hird et al published on Jun. 16, 2011, US 2011/0139657A1 Hird et al published on Jun. 16, 2011, US 2011/0152812A1 Hird et al published on Jun. 23, 2011, US 2011/0139662A1 Hird et al published on Jun. 16, 2011, and US 2011/0139659A1 Hird et al published on Jun. 16, 2011. These components include, but are not limited to, topsheet nonwovens, backsheet films, backsheet nonwovens, side panel nonwovens, barrier leg cuff nonwovens, superabsorbent, nonwoven acquisition layers, core wrap nonwovens, adhesives, fastener hooks, and fastener landing zone nonwovens and film bases. In at least one embodiment, a disposable absorbent article component comprises a bio-based content value from about 10% to about 100% using ASTM D6866-10, method B, in another embodiment, from about 25% to about 75%, and in yet another embodiment, from about 50% to about 60% using ASTM D6866-10, method B. In order to apply the methodology of ASTM D6866-10 to determine the bio-based content of any disposable absorbent article component, a representative sample of the disposable absorbent article component must be obtained for testing. In at least one embodiment, the disposable absorbent article component can be ground into particulates less than about 20 mesh using known grinding methods (e.g., Wiley® mill), and a representative sample of suitable mass taken from the randomly mixed particles.

### Measurement

### 1. Basis Weight Test

The basis weights of materials are measured according to WSP 604.0 (08). All measurements are performed in a laboratory maintained at 23° C.±2 C.° and 50%±2% relative humidity and test specimens are conditioned in this environment for at least 2 hours prior to testing. To obtain the nonwoven sample, cut a rectangle-shaped nonwoven specimen from the article with an area of 100 cm² (for example, 100 mm×100 mm), and measure its basis weight following the measurement principle used by the standard method above. At least 5 replicates for the test specimen are tested and the average value of the at least five replicates is reported to the nearest 1 gsm (g/m2) as the basis weight of the test specimen.

### 2. Thickness Test

The thickness of a test specimen is measured as the distance between a reference platform on which the specimen rests and a pressure foot that exerts a specified amount of pressure onto the specimen over a specified amount of time. All measurements are performed in a laboratory maintained at 23° C.±2 C.° and 50%±2% relative humidity and test specimens are conditioned in this environment for at least 2 hours prior to testing.

Thickness is measured with a manually-operated micrometer equipped with a pressure foot capable of exerting a steady pressure of 1.45 kPa (14.7 g/cm²)+0.01 kPa onto the test specimen. The manually-operated micrometer is a dead-weight type instrument with readings accurate to 0.01 mm. A suitable instrument is Frank Type 16502.F000 form Frank Prüfgeräthe GmbH or equivalent. The pressure foot is a flat ground circular movable face with a diameter that is smaller than the test specimen and capable of exerting the required pressure. A suitable pressure foot has a diameter of 56.4 mm, however a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Obtain a test specimen by removing it from an absorbent article, if necessary. When excising the test specimen from an absorbent article, use care to not impart any contamination or distortion to the test specimen layer during the process. The test specimen is obtained from an area free of folds or wrinkles, and it must be larger than the pressure foot.

To measure thickness, first zero the micrometer against the horizontal flat reference platform. Place the test specimen on the platform with the test location centered below the pressure foot. Gently lower the pressure foot with a descent rate of 3.0 mm+1.0 mm per second until the full pressure is exerted onto the test specimen. Wait 5 seconds and then record the thickness of the test specimen to the nearest 0.01 mm. In like fashion, repeat for a total of ten replicate test specimens. Calculate the arithmetic mean for all thickness measurements and report as Thickness to the nearest 0.01 mm.

The thickness factor is the thickness per 10 gsm of basis weight of the sample. So, the equation is thickness/(basis weight/10).

### 3. Tensile Strength Test

(1) The CD tensile strength of a specimen is measured according to WSP 110.4-09 with conditions below.
Preload: 0.1N
Test Speed: 100 mm/min
Sample Width: 50 mm
Sample length: sufficiently longer than gauge length
Gauge Length: 100 mm

A CD tensile factor is the CN tensile per 10 gsm of basis weight of the sample. So, the equation is CN tensile/(basis weight/10).

(2) MD tensile strength of a specimen is measured according to WSP 110.4-09 with conditions below.
Preload: 0.1N
Test Speed: 100 mm/min
Sample Width: 50 mm
Sample length: sufficiently longer than gauge length
Gauge Length: 100 mm

### 4. Fiber Diameter Test

### (1) Sample Conditioning and Specimen Preparation

When a nonwoven is available in a raw material form, a rectangular test sample with a size of 310 mm×90 mm is cut from the raw material. When a nonwoven is a component of a finished product, the nonwoven is removed from the finished product using a razor blade to excise the nonwoven from other components of the finished product to provide a nonwoven test sample around the outer perimeter of a 5±1 cm×5±1 cm area. (If the nonwoven is of insufficient size to permit a 5±1 cm×5±1 cm area to be excised from the finished product, the largest square of nonwoven that can be extracted is excised and used as the specimen henceforth). As necessary, cryogenic spray (such as Cyto-Freeze, Control Company, Houston TX) can be used to remove the nonwoven from the underling layer.

To prepare the specimen for cross-section imaging, submerge the nonwoven removed from the finished product in liquid nitrogen and use a razor blade to cut a 10 mm×4 mm specimen from the nonwoven. The specimen is mounted vertically on a sample stage with the wearer facing side attached onto the sample using carbon tape. The cross-sectional edge of the specimen is facing upwards and oriented such that it is substantially aligned to the horizontal direction for subsequent imaging. The specimen is sputtered with platinum to avoid electric charging and improve overall conductivity, under the conditions of 30 mA current and 120 second coating time.

### (2) Measurement of Fiber Diameter

Cross section images of specimen are taking using a Scanning Electron Microscope (SEM) such as Tabletop Microscope TM3000 (Hitachi, Japan), or equivalent. The platinum-coated specimen is subsequently transferred into the SEM specimen vacuum chamber for the imaging analysis. An appropriate magnification and working distance are chosen so that the cross-section specimen is suitably enlarged for fiber diameter measurement and imaged under an acceleration voltage of 5 kV. The specimen images are saved as 8-bit jpeg images containing a linear distance scale for calibration. Measurement of the fiber diameter is performed using an image analysis program such as ImageJ software (version 1.52p or above, National Institutes of Health, USA) or equivalent. Record the values of fiber diameter to the nearest 0.1 micron (as shown in FIGS. 3A-3C). Measure at least 10 replicates and report the average value to the precision described above.

### 5. Cuff Tearing Force Test

Cuff tearing force is measured by a tensile measurement tester such as MTS Criterion model 42 from MTS System.

### (1) Specimen Preparation

Referring to FIG. 9, open a diaper 20 and mount it on a sample stage with the wearer facing side attached on the sample stage using a carbon tape. Draw the line A 150 mm above from the transversal centerline 90 to parallel with the transversal centerline 90. Draw the line B 150 mm below from the transversal centerline 90 to parallel with the transversal centerline. Using a precision cutter, cut the diaper along with line A and line B to obtain a specimen with a length of 30 mm in the diaper length direction and a width of a full diaper width.

### (2) Cuff Tearing Force Measurement

Fix the specimen on the tensile measurement tester, in such a way each of the left and right sides of the specimen is fixed with a clamp. The cuff tearing force is measured according to WSP 110.4-09 with conditions below.
Preload: 0.1N
Test Speed: 500 mm/min
Sample Width: 30 mm
Sample length: full diaper width
Gauge Length: 150 mm

### 6. Back Ear Tearing Force Test

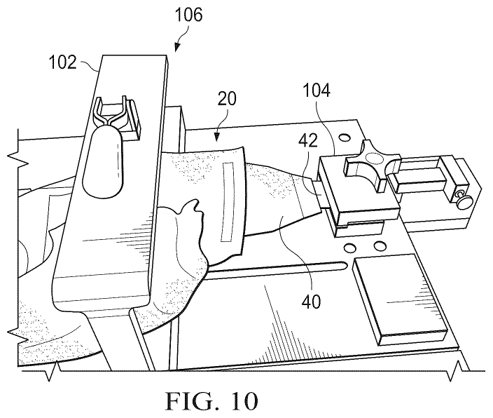

The back ear peak force is measured by a tensile measurement tester such as Work to Yield Primary Peak Force Tester, Model 1000, available from ChemInstruments (Ohio, USA) with a stationary clamp, such as a flat bar clamp, for example R-WTY Flat Bar Grip or equivalent, or a round bar clamp for example R-WTY Round Bar Grip or equivalent. They are available from ChemInsturments (Ohio, USA). Test procedure is described as below referring to FIG. 10.
1) Open and unfold a sample product, a taped diaper 20 in this case, and slide it into the sled clamp 102 in the tester 106. Tighten and lock the sled clamp 102.
2) Open the stationary clamp 104. Open the tape tap 42 in the diaper 20 completely and insert the tape tap 42 into the stationary clamp 104. Close and tighten the stationary clamp 104.
3) Make sure the ear 40 and tape tab 42 are straight, and perpendicular to both of the sled clamp 102 and stationary clamp 104.
4) Flatten the ear 40 but leaving it slightly loose by sliding the sled clamp 102 away from the stationary clamp 104.
5) Start the test by turn on the testing system by moving the sled clamp 102 away from the stationary clamp 104. Sled clamp 102 is moved in a speed of 178 cm/min.
6) Record the peak force nearest to 0.01 Newton.

### 7. Fiber Orientation Disorder Level

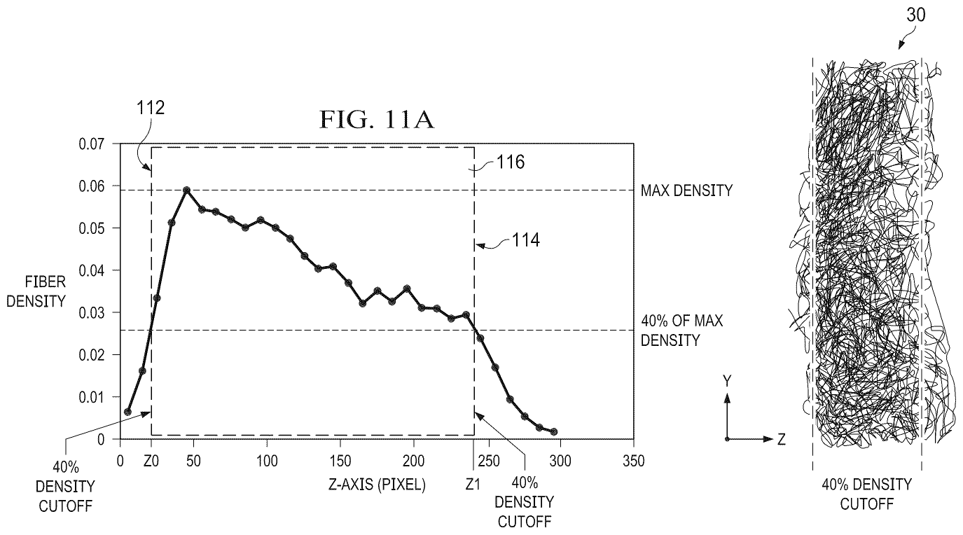

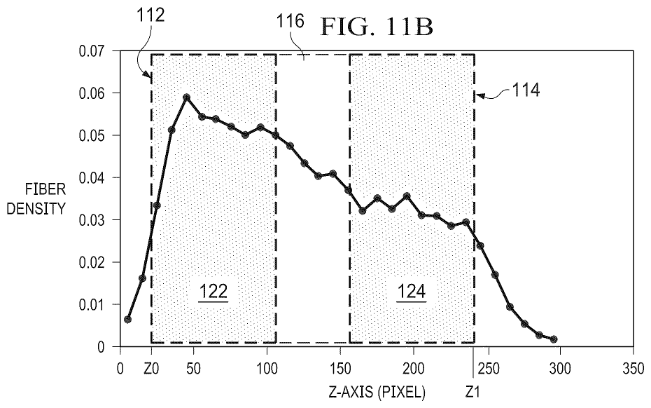

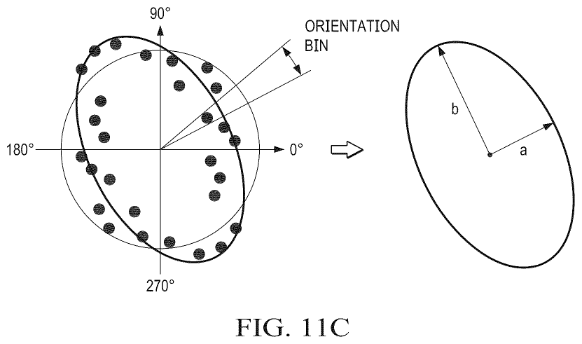

The nonwoven fiber orientation disorder level is characterized based on a micro-CT image with a resolution of 5 μm or better. Referring to FIG. 11A, with the micro-CT image data set, the nonwoven structure is aligned horizontally with the z direction as the thickness direction of a sample nonwoven 30. In nonwoven 30, nonwoven edges in z direction are determined according to fiber density distribution. (The x and y directions are perpendicular to each other and to the z direction. They are assigned arbitrarily but held constant throughout this method.) The density profile along z-axis is calculated by division the nonwoven 30 into adjacent slices with z direction thickness of 0.1 millimeter. For each slice the fiber density is calculated. The z-axis value corresponding to 40% of the maximum value of fiber density (first appears) in each (surface) side of the fiber density distribution curve is defined as z0 and z1 with z1>z0. That is, each of z0 and z1 indicate 40% density cut off point which determines 40% cut off lines 112 and 114. The area between the 40% cut off lines 112 and 114 is defined as Main Fiber Zone 116. where fiber orientation disorder levels are measured. The thickness of the Main Fiber Zone is Δz=z1−z0. Fibers outside the Main Fiber Zone are omitted from further analysis.

Referring to FIG. 11B, two orientation calculation zones, a first zone 122 and a second zone 124 are defined in the Main Fiber Zone. The first zone 122 is the region with z-value of (z0≤z≤(z0+40% Δz)) while the second zone 124 is the region with ((z1−40% Δz)≤z≤z1). Fiber orientation disorder level in each of the first and second zones are calculated according to the following steps.

For each of the first and second zones 122, 124, fibers are segmented to segments of chord length of 0.04 millimeter along the curvilinear path. For each segment, orientation in XY plane is calculated from the XY coordinates of both the segment ends (Z coordinate is not used) as an angle of the segment projection in XY plane with the X axis, ranging from 0 to 180 degree. The histogram of orientation angle of the fiber segments in XY plane is then calculated with bins in 10-degree increments in the range of 0 to 180 degrees.

As the segment orientation in XY plane is bidirectional, an entire 360-degree distribution is defined by simply repeating the same fiber-angle segment count histogram in step 1 between 180 and 360 degrees. The histogram is plotted on polar coordinates with angle corresponding to fiber segment bin angle and radius proportional to fiber segment count in each angular bin. An ellipse centered at origin is then fitted using least squares fitting method. Referring FIG. 11C, the ratio of the minor axis a and major axis b of the fitted ellipse is calculated with an accuracy of four decimal places and recorded as the fiber orientation disorder level for each zone 122, 124.

Between the fiber orientation disorder levels in the first zone 122 and the second zone 124, the lower fiber orientation disorder level is reported as a fiber orientation disorder level for the first layer, i.e., first FODL; and the higher fiber orientation disorder level is reported as a fiber orientation disorder level for the second layer, i.e., second FODL.

For each sample nonwoven, five like replicates are analyzed via micro-CT, and fiber orientation disorder levels in the first calculation zone 122 and the second calculation zone 124, and the fiber orientation disorder level difference between them are calculated as described above. The average values of the five replicates are reported as the fiber orientation disorder levels in zones 122 and 124, and fiber orientation disorder level difference of the test sample.

### EXAMPLES

### Example 1: Nonwoven Preparation

Various nonwoven substrates were prepared. Nonwovens 1, 2 and 4 are materials available from market. Nonwovens 3, 5 and 6 were produced as below. All fibers that make up the nonwovens are sheath/core type bicomponent fibers.

Nonwoven 1: FJ200 from Dayuan (China) made from 2denier PE/PET fibers. 19 gsm carded air-through nonwoven.

Nonwoven 2: N_MC1003 from Wisdom (China) made from 2denier PE/PET fibers. 16 gsm carded air-through nonwoven.

Nonwoven 3: Nonwoven having a 8 gsm first layer and a 8 gsm second layer, both of the first and second layers being composed of 60% 2denier PE/PET fibers and 40% 1.5denier PE/PET, fibers was produced by a conventional single carding process having one cylinder and two doffers. The second doffer speed was slower than the first doffer speed, so that the fibers in the second stratum were more disordered, i.e., more fibers in the second stratum were in CD orientation while fibers in the first stratum are more in MD orientation.

Nonwoven 4: N_HB2008 from Wisdom (China). 20 gsm carded air-through nonwoven having a 10 gsm first layer of 100% 0.8deneir PE/PET fibers, and a 10 gsm second layer of 100% 2denier PE/PET fibers.

Nonwoven 5: A 20 gsm carded air-through nonwoven having a 10 gsm first layer of 100% 0.8denier PE/PET fibers, and a 10 gms second layer of 50% 2denier PE/PET fibers and 50% 1.5deneir PE/PET fibers was produced using a conventional double carding process having two serial carding machines. The first and second layers were formed from the first and second carding machines, respectively, and layered together in a face to face relationship before heat-bonding. The fiber condensing level in the first layer was lower than the fiber condensing level in the second layer, so that the FODL of the first layer was lower than the FODL in the second fiber layer.

Nonwoven 6: A 20 gms carded air-through nonwoven was produced by a conventional single carding process having one cylinder and two doffers using 1.5d PE/PET fibers. The two doffers were operated in the same speed.

### Example 2: Nonwoven Properties

Properties of nonwovens prepared in Example 1 were tested according to test methods disclosed herein and are displayed in Table 1 below. Basis weight values of nonwovens were measured according to the Basis Weight Test. Thickness values and fiber diameter values of nonwovens were measured according to the Thickness Test and Fiber Diameter Test, respectively. SEM photos of FIGS. 3A-3C of nonwoven 3, and SEM photos of FIGS. 4A-4C of nonwoven 5 were taken during the process of these measurements. In FIG. 3B and FIG. 3C, fiber diameters of 1.5denier PE/PET fibers and 2denier PE/PET fibers were measured, respectively. In FIG. 4B, fiber diameters of 0.8 denier PE/PET fibers were measured. In FIG. 4C, fiber diameters of 2 denier PE/PET fibers (top image) and 1.5denier PE/PET fibers (bottom image) were measured.

MD Tensile and CD Tensile values of nonwovens were measured according to Tensile Strength Test.

For nonwoven 3, 5 and 6, fiber orientation disorder levels in first layers and second layers of the nonwovens, and differences of fiber orientation disorder levels in the first and second layers were measured according to Fiber Orientation Disorder Level Test.

Nonwovens 3 and 5 are embodiments of nonwoven webs of the present disclosure, while nonwovens 1-2, 4 and 6 are provided merely for comparison purposes.

TABLE 1

| Nonwoven | 1 | 2 | 3 | 4 | 5 | 6 |
|---|---|---|---|---|---|---|
| Basis weight (g/m²) | 19.3 | 16.2 | 16.0 | 19.5 | 19.6 | 20.7 |
| Thickness (mm) | 0.50 | 0.36 | 0.53 | 0.42 | 0.52 | 0.35 |
| Thickness factor | 0.26 | 0.22 | 0.33 | 0.21 | 0.27 | 0.17 |
| MD Tensile (N/5 cm) | 29.5 | 25.2 | 26.8 | 31.1 | 31.6 | 35.7 |
| CD Tensile (N/5 cm) | 5.5 | 5.1 | 6.2 | 6.7 | 8.2 | 6.0 |
| CD Tensile factor | 0.311 | 0.314 | 0.388 | 0.344 | 0.418 | 0.290 |
| First FODL | | | 0.324313 | | 0.219182 | 0.232178 |
| Second FODL | | | 0.407879 | | 0.31426 | 0.266882 |
| Delta FODL | | | 0.0836 | | 0.0951 | 0.0347 |

Nonwoven 3 exhibits parity or high thickness and CD tensile compared to nonwoven 1 despite a lower basis weight, and exhibits much higher thickness and CD tensile compared to nonwoven 2 without basis weight increase. Nonwoven 5 exhibits much higher thickness and CD tensile compared to nonwovens 4 and 6 without basis weight increase.

Both nonwovens 3 and 5 have increased thickness factor and CD tensile factor compared to nonwovens 1, 2, 4 and 6.

### Example 3: Diaper Preparation

Taped diapers as exemplary absorbent articles were obtained as described below.

Diaper 1: Pampers Qingxinbang Taped size L (Procter and Gamble China) having nonwoven 1 as an outer cover.

Diaper 2: Pampers Qingxinbang Taped size L (Procter and Gamble China) based, replacing the outer cover with Nonwoven 3

Diaper 3: Pampers Yijibang Taped size L (Procter and Gamble China)

Diaper 4: Pampers Yijibang Taped size L (Procter and Gamble China) based, replacing the outer cover with Nonwoven 5

### Example 4: Cuff Tearing

Diapers 1 and 2 were tested on cuff tearing according to the Cuff Tearing Test. Results are indicated in Table 2 below. Diaper 2 exhibits a significantly high cuff tearing force compared to Diaper 1.

TABLE 2

| Sample | Cuff tearing force, N/3 cm |
|---|---|
| Diaper 1 | 7.1 |
| Diaper 2 | 8.4 |

### Example 5: Back Ear Tearing

Diapers 3 and 4 were tested on back ear tearing according to the Back Ear Tearing Test. Results are indicated in Table 3 below. Diaper 4 exhibits a significantly increased back ear tearing force compared to Diaper 3.

TABLE 3

| | Back ear peak force | Back ear peak force |
|---|---|---|
| Sample | (Drive side*), N | (Operation side*), N |
| Diaper 3 | 31.9 | 35.0 |
| Diaper 4 | 33.0 | 38.2 |

Drive side*: left side of a diaper when worn on a baby
Operation side*: right side of a diaper when worn on a baby

### Example 6: Consumer Acceptance Test

5 sensory panelists who were qualified on softness sensory evaluation method. Five (5) diapers of each of diaper 1 and diaper 2 were provided to the panelists to touch and feel the outer cover covered area of the diapers with their hands one by one. Each respondent was asked to fill in a questionnaire individually after touching the test sample one by one. In the questionnaire, there were items including the 5 items as found in Table 4 below, and each respondent was requested to sort and rate the test samples against those values using the ratings from 1 to 10, which were scored as such: ‘1=poor, 10=excellent’. The scores were averaged.

TABLE 4

| | Diaper 1 | Diaper 2 |
|---|---|---|
| Drapability | 8.0 | 8.0 |
| Less grainy feel | 5.4 | 5.4 |
| BS Slipperiness | 8.3 | 8.2 |
| Crotch Flexibility | 7.2 | 7.2 |
| Leg Gather Less | 7.8 | 7.8 |
| Harshness | | |

Diaper 2 even with a lower basis weight outer covert exhibits parity overall softness performance to Diaper 1.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A nonwoven substrate comprising a first surface, an opposite second surface, a first layer forming the first surface, and a second layer located below the first layer in a direction of a thickness of the nonwoven substrate;
wherein the first layer comprises first fibers and the second layer comprises second fibers, the first fibers and the second fibers comprising staple heat-fusible fibers;
wherein the first layer has a first fiber orientation disorder level, and the second layer has a second fiber orientation disorder level higher than the first fiber orientation disorder level, as measured according to Fiber Orientation Disorder Level Test; and
wherein the difference between the first and second fiber orientation disorder levels is higher than about 0.05.

2. The nonwoven substrate according to claim 1, wherein the second fiber orientation level is not lower than about 0.27.

3. The nonwoven substrate of claim 1, wherein the nonwoven substrate has a unitary structure.

4. The nonwoven substrate of claim 1, wherein the nonwoven substrate is a carded air through nonwoven.

5. The nonwoven substrate of claim 1, wherein the first fibers and the second fibers are different fibers.

6. The nonwoven substrate of claim 1, wherein the first fibers have a diameter no greater than about 20 μm.

7. The nonwoven substrate of claim 1, wherein the second fibers have a diameter no less than about 12 μm.

8. The nonwoven substrate of claim 1, wherein the first fibers have a diameter smaller than a diameter of the second fibers.

9. The nonwoven substrate of claim 1, wherein the first fibers and the second fibers are the same fibers.

10. The nonwoven substrate of claim 1, wherein the first layer comprises third fibers.

11. The nonwoven substrate of claim 1, wherein the second layer comprises fourth fibers, and wherein the fourth fibers have a diameter smaller than a fiber diameter of the second fibers.

12. The nonwoven substrate of claim 1, wherein the nonwoven substrate has a basis weight no higher than about 45 gsm.

13. The nonwoven substrate of claim 1, wherein the nonwoven substrate has a CD tensile factor no less than 0.35.

14. The nonwoven substrate of claim 1, wherein the nonwoven substrate has a CD tensile strength no less than about 5.5N/5 cm as measured by Tensile Strength Test.

15. The nonwoven substrate of claim 1, wherein the nonwoven substrate has a thickness factor no less than about 0.25.

16. An absorbent article comprising a wearer-facing surface and a garment-facing surface, and comprising;
a liquid pervious topsheet;
a liquid impervious backsheet;
an absorbent core disposed between the topsheet and the backsheet; and
a nonwoven substrate according to claim 1.

17. The absorbent article of claim 16, wherein the absorbent article comprises an outer cover attached to the backsheet to form at least part of the garment-facing surface of the absorbent article, the outer cover comprising the nonwoven substrate such that the first layer of the nonwoven substrate forms the garment-facing surface of the absorbent article.

18. The absorbent article of claim 16, wherein the liquid pervious topsheet comprises the nonwoven substrate such that the first layer forms the wearer-facing surface of the absorbent article.

19. An absorbent article comprising a wearer-facing surface and a garment-facing surface, and comprising a front elastic belt region, a back elastic belt region, a crotch region, and a pair of side seams which join the front elastic belt region and the back elastic belt region to form a waist opening and a pair of leg openings, the crotch region extending longitudinally between the front elastic belt region and the back elastic belt region;
wherein each of the front and back elastic belt regions comprise a laminate comprising an inner belt sheet, an outer belt sheet, and a plurality of elastic members running in the transverse direction;
wherein the outer belt sheet comprises the nonwoven according to claim 1 such that the first layer of the nonwoven substrate forms the garment-facing surface of the absorbent article.
